(19) United States
(12) Patent Application Publication
(10) Pub. No.: US 2022/0273220 A1
(43) Pub. Date: Sep. 1, 2022

(54) IMPLANT, ENSEMBLE COMPRISING SUCH AN IMPLANT AND METHOD FOR FABRICATING SUCH AN IMPLANT

(71) Applicants: INSTITUT NATIONAL DE LA SANTE ET DE LA RECHERCHE MEDICALE(INSERM), Paris (FR); UNIVERSITE GRENOBLE ALPES, Saint Martin d'Heres (FR); UNIVERSITE GUSTAVE EIFFEL, Champs-Sur-Mame (FR)

(72) Inventors: Fannie DARLOT, Saint Martin d'Heres (FR); Gaëlle OFFRANC PIRET, Saint Martin d'Heres (FR); Lionel ROUSSEAU, Noisy le Grand (FR); Paul VILLARD, Saint Martin d'Heres (FR)

(21) Appl. No.: 17/626,254

(22) PCT Filed: Jul. 15, 2020
(86) PCT No.: PCT/EP2020/070035

§ 371 Date: Jan. 11, 2022

(30) Foreign Application Priority Data
Jul. 15, 2019 (EP) 19305942.5

(51) Int. Cl. A61B 5/293; A61B 5/37; A61B 5/00

(52) U.S. Cl. CPC A61B 5/293; A61B 5/37; A61B 5/686; A61B 2562/0209; A61N 1/0531

(57) ABSTRACT

The present invention relates to an implant adapted to be implanted at least partially in a biological tissue (20). Today's implants remain very susceptible to mechanical damage. The inventors have thus developed an implant having a greater reliability in terms of resistance to mechanical stress than existing implants, while allowing for easy connection with different parts of a biological tissue. This implant comprises an implant body (30) and a set of electrically conductive wires (55), each wire (55) comprising a first portion (65) electrically connected to the body (30), a second portion (70) and a third portion (75) intended to be electrically connected to the tissue (20), The implant (10) comprises a set of arms (25) comprising each an insulating sheath (60) and a bundle (52) of wires (55), each bundle (52) comprising at least two subsets (62) of wires (55).

The present invention concerns an implant and an ensemble comprising such an implant. The present invention also concerns a method for fabricating such an implant.

Implants of many types are used to carry electrical impulses in the body of a human or animal patient. Some are used to carry these impulses from one part of the body to another, acting as substitute for severed nerves. Some other implants are used to carry electrical signals between an organ of the body, such as the brain, notably the brain cortex, and an outside apparatus. This apparatus is, notably, meant to record the activity of the corresponding organ, or to command a stimulation of the organ, through the implant.

In this view, many implants comprise a set of electrodes. Each electrode is a flexible conductive wire, one extremity of which is meant to be electrically connected to the organ, while the rest of the electrode is encased in an insulating bulk so that only the electrode's extremity is in electrical contact with the patient's body. Each electrode is attached to an electrical connector, allowing for the implant to be electrically linked to an outside apparatus, so as to permit the transfer of electricity between the apparatus and the organ.

The electrodes have a very small diameter in order to record the activity of (or electrically stimulate) a specific part of the organ such as a single neuron. Implants composed of those small diameter electrodes have reduced dimensions and are very susceptible to mechanical stress due to the movement of the body (notably the heart beating, head movements or movements of an inferior and superior member). There is a balance to find so that their fabrication, handling and resistance to sterilization process, necessary prior to their implantation, are feasible while their dimension and characteristics will approach the one of tissue cells, naturally present in the organ to be implanted. The other fragility of those implants is that they are susceptible to chemical corrosion in the biological tissue they are placed in. Thus, the implant's small dimensions, these movements and this corrosion can affect the stability of the recording or stimulation, as the recorded or stimulated area can detach from the electrode for instance. They can also damage the implant and thus affect the quality of the recording or stimulation or even prevent recording or stimulating the target area altogether. Consequently, it is usually preferable to have several electrodes so that in case one electrode is damaged, other neighboring electrodes can be relied upon to provide an electrical connection to the same area of the organ. However, it remains usually preferable that the same electrode of an implant remains attached to a same area, for example to a same neuron when the target area is a part of an animal's brain. In that sense, a good adhesion of the implant to the tissue is crucial.

Today's implants remain very susceptible to mechanical and chemical corrosion damages. In addition, those implants are not suited for recording (or stimulating) different portions of a same organ, as all electrodes of the bundle are usually electrically connected to a same area, mostly to ensure redundancy within that same area. In order to interface electrodes with different parts of the organ, several implants must be used, which in turn increases the complexity and cost of the implantation process.

Document US 2014/0277258 A1 discloses several examples of implants, including examples wherein electrodes extend from circular “surface probes”.

Document US 2019/150774 A1 discloses an electrode device comprising a macroelectrode and microelectrodes encompassed within the macroelectrode.

Document CN 106 178 259 A discloses an implant having electrodes distributed on both sides of a symmetry line of the implant.

The article “Implantable neurotechnologies: a review of micro-nanoelectrodes for neural recording”, by Patil Anoop C et al, published in “Medical and biological engineering and computing” vol. 54, no. 1, pages 23-44 on 11 Jan. 2016, discloses various examples of electrodes for neural recording.

Document U.S. Pat. No. 6,091,979 A discloses an electrode array for cranial implantation comprising electrode cables.

US 2005/154435 A1 discloses an electrode assembly comprising a set of paddles, each paddle comprising a set of electrical contacts.

US 2018/303595 A1 discloses a medical device comprising at least one electrode.

Document US 2018/345019 A1 discloses a neural interface system comprising an implanted portion including electrodes.

There is therefore a need for an implant, having a greater reliability in terms of resistance to mechanical stress (with a good compromise for the steps of implant fabrication, implant sterilization, implant insertion in the biological tissue, and implant adhesion to the tissue) and chemical corrosion, than existing implants, while allowing for easy connection with different parts of a patient's organ.

In this view, the present specification concerns an implant, adapted to be implanted at least partially in a biological tissue of an animal, comprising an implant body and a set of electrically conductive wires, each conductive wire comprising a first portion electrically connected to the implant body a second portion and a third portion intended to be electrically connected to the biological tissue, the second portion being interposed between the first portion and the third portion, the implant comprises a set of arms comprising each an electrically insulating sheath and a bundle of said conductive wires, each bundle comprising at least two subsets of conductive wires, each subset comprising at least two conductive wires each sheath having a single proximal portion, a set of middle portions and a set of distal portions, each middle portion corresponding to a subset of conductive wires, each distal portion corresponding to a single conductive wire, the proximal portion of a sheath extending from the implant body and encasing the first portion of each conductive wire of the bundle, each middle portion of the sheath extending from the proximal portion and encasing the second portion of each conductive wire of the corresponding subset, and each distal portion of the sheath extending from a middle portion and encasing the third portion of a single conductive wire of the bundle.

Since the distal portions of each arm's sheath are connected in groups to corresponding middle portions of the sheaths, these middle portions being themselves connected to a same proximal portion, the mechanical resistance of the implant is augmented while still allowing for an important degree of flexibility, since the middle portions can move relative to each other, as can the distal portions, so as to accommodate relative movements of different parts of the biological tissue. For these reasons, the risk of mechanical failure of the implant or of disconnection of an electrode from the surrounding tissue is limited, as the end (third) portions of the wires can follow the tissue's movement independently from each other, while the first and second portions of the wires are regrouped in a stronger sheath than they would be were the wires' sheaths separate along their whole length.

In the invention, the use of three different levels of separation of the arms (one single proximal portion and several middle portions, themselves separating each into several distal portions) enable a progressive increase in flexibility from the base to the tip of the arm while keeping the arm's base (corresponding to the proximal portion) very resilient.

In notable contrast, since the electrodes of document US 2014/0277258 A1 extend from individual circular “surface probes”, which are not themselves connected to a single portion of an arm of the implant, the resulting implant only shows two such different levels of separation. Therefore, the implant according to the invention enables a better balance between resilience and flexibility than that of US 2014/0277258 A1.

According to specific embodiments, the implant comprises one or several of the following features, according to any possible combination:
each arm extends from the implant body along a main direction, the main directions of the arms being coplanar.
each arm extends radially from the body, an angle between the main directions of a pair of successive arms being notably equal for each pair of successive arms.
the implant body comprises a main portion and an attachment portion, the attachment portion extending from the main portion, each arm extending from the attachment portion, the attachment portion being shaped as circular sector.
each arm comprises at least three subsets of conductive wires, each subset comprising at least three conductive wires.
a length of the proximal portion, a length of the middle portion and a length of the distal portion of each sheath are each comprised between 30 percent and 40 percent of a total length of the arm.
at least one of the following properties is verified:
   each conductive wire of at least one subset is electrically connected to at least one other conductive wires of the subset, notably by at least one electrical conductor linking the third portions of the conductive wires;
   the distal portion of at least one sheath is linked to a distal portion of at least one other sheath of the same subset by a linking portion of the sheath, the linking portion being fixed to both distal portions linked by the linking portion;
   a face of at least one conductive wire or of at least one sheath is nanostructured.
each conductive wire comprises a metallic film, the thickness of the film being notably comprised between 1 nanometers and 3000 nanometers.
the implant comprises an electrical connector configured to be electrically connected to a device distinct from the implant body, the implant body comprising electrical conductors configured to connect each conductive wire to the electrical connector.
the implant further comprises an extension piece comprising a substrate and conductive lines supported by the substrate, the substrate comprising a first extreme portion, a second extreme portion and an intermediate portion interposed between the first extreme portion and the second extreme portion, the extension piece being connected to the electrical connector at the first extreme portion, each conductive line being configured to carry an electrical current between the first extreme portion and the second extreme portion, a thickness of the intermediate portion being inferior or equal to the thickness of the first extreme portion and inferior or equal to the thickness of the second extreme portion.
the implant body is integral with the arms.
at least one conductive wire of one arm is electrically connected, through the implant body to a conductive wire of another arm.

The present specification also concerns an ensemble comprising a implant as previously defined and an instrument comprising a set of tools, the set of tool comprising a first tool able be fixed to the implant body and, for each arm, a second tool configured to maintain the arm in a predefined position relative to the implant body.

The present specification also discloses a method for fabricating an implant as previously defined, wherein each sheath is made of an electrically insulating first material, the method comprising:
a step for fabricating a first layer made of the first material,
a step for depositing, onto the first layer, at least one second layer of an electrically conductive second material to form the conductive wires, and,
a step for depositing, onto the first and second layers a third layer of the first material so as to form each sheath.

According to a specific embodiment, the method further comprises a step for etching away part of the third layer so as to define, for each conductive wire, an opening for electrically connecting the conductive wire to the cortex, the part of the third layer being etched away using notably an ion beam.

The present specification also concerns a method for implanting an implant, comprising a step for implanting, in a biological tissue of an animal, at least one implant as previously defined.

Features and advantages of the invention will appear upon reading the following specification, given only as a non-limiting example, and made with reference to the associated drawings, in which:

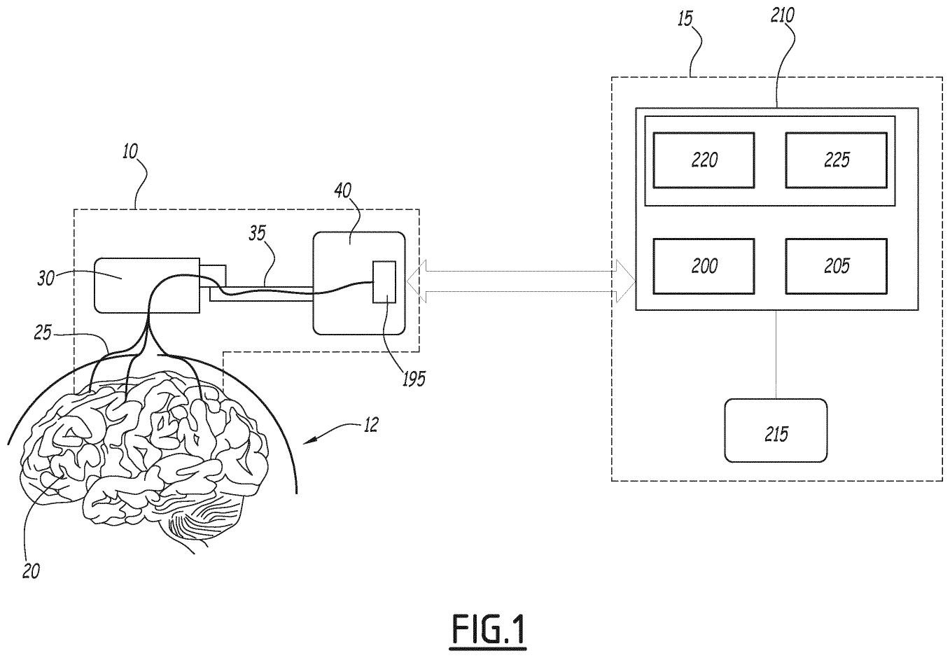

FIG. 1 is a schematics of an ensemble comprising an implant and a distant apparatus,

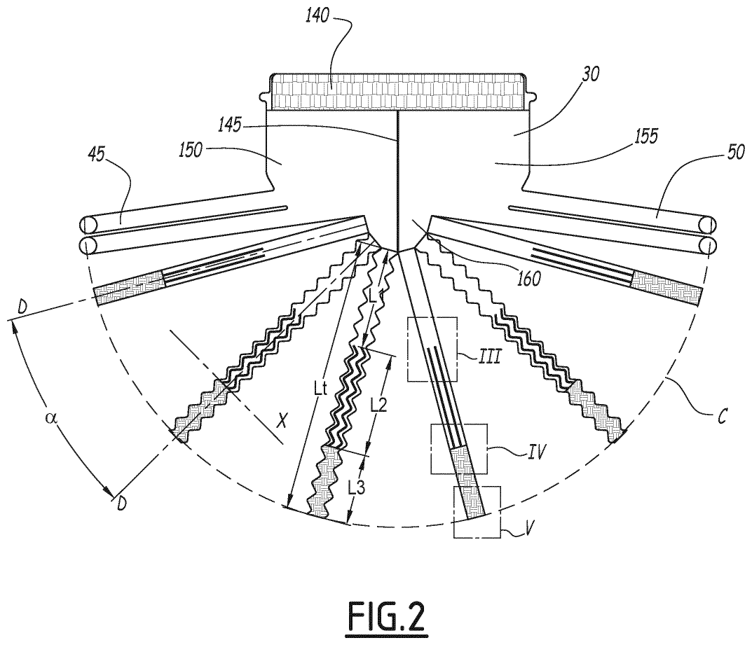

FIG. 2 is a schematics of an implant according to the invention, comprising a set of electrodes and an electrical connector,

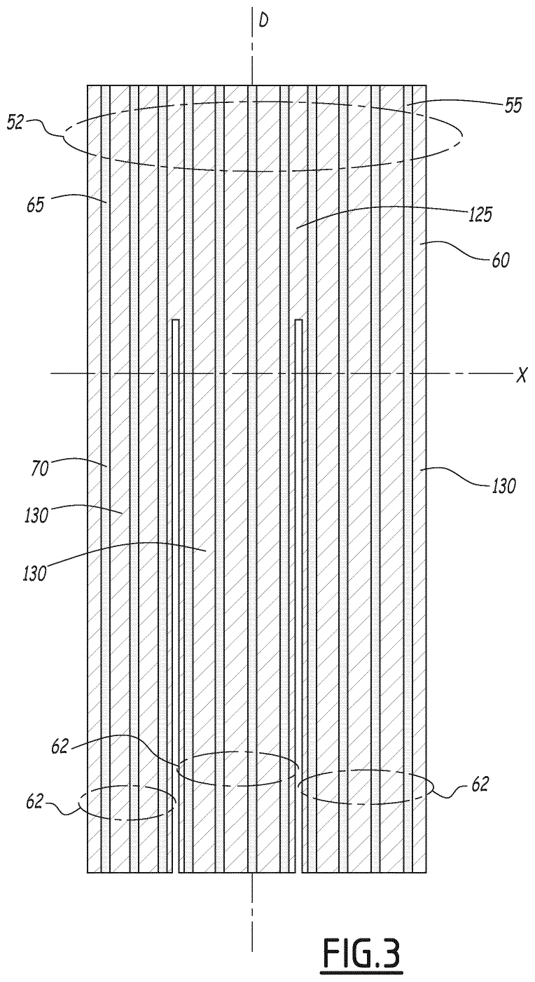

FIG. 3 is a zoom of the area III of FIG. 2,

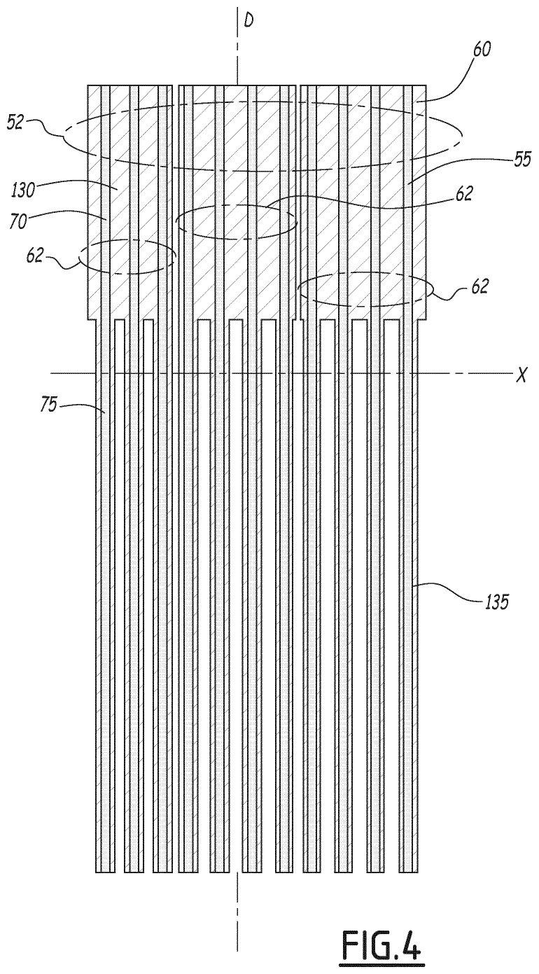

FIG. 4 is a zoom on the area IV of FIG. 2,

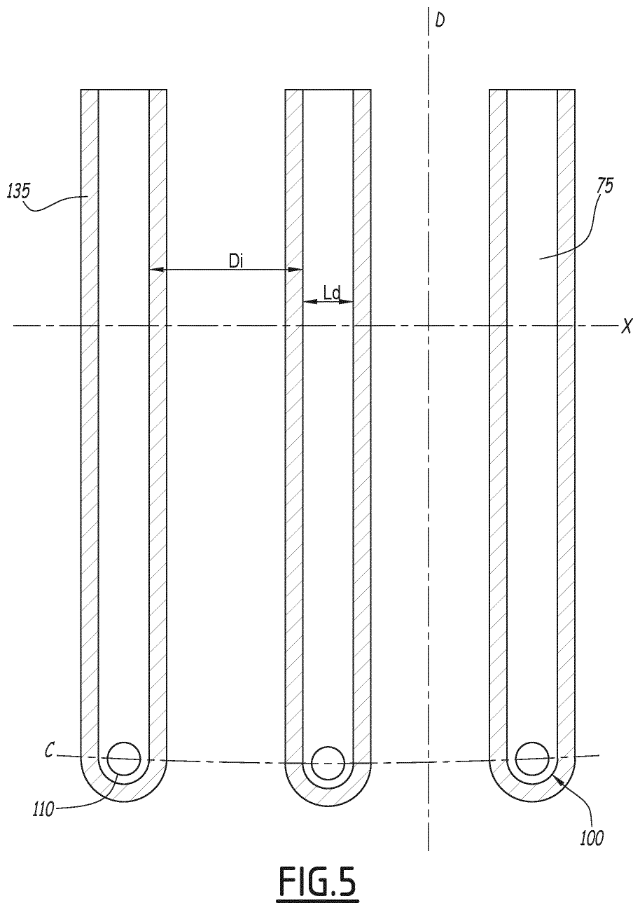

FIG. 5 is a zoom on the area V of FIG. 2, showing an extremity of a set of electrodes,

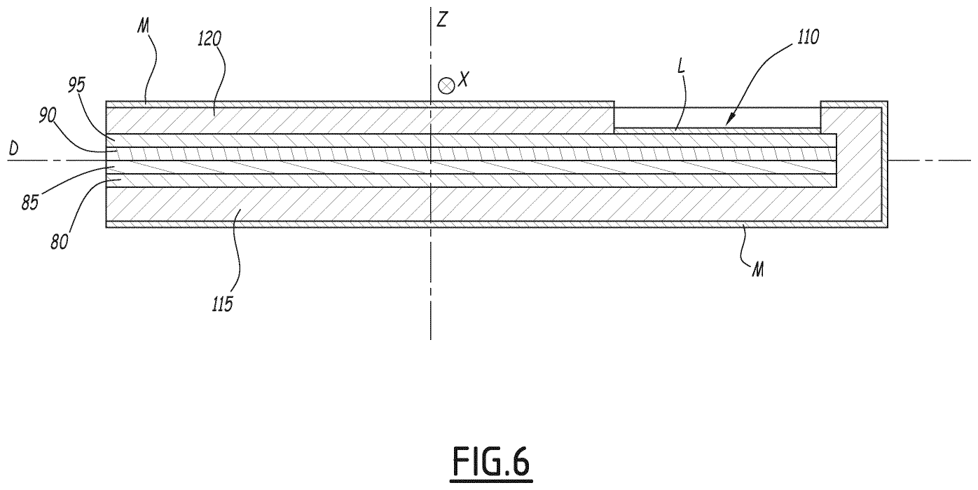

FIG. 6 is a cut-away side view of an electrode,

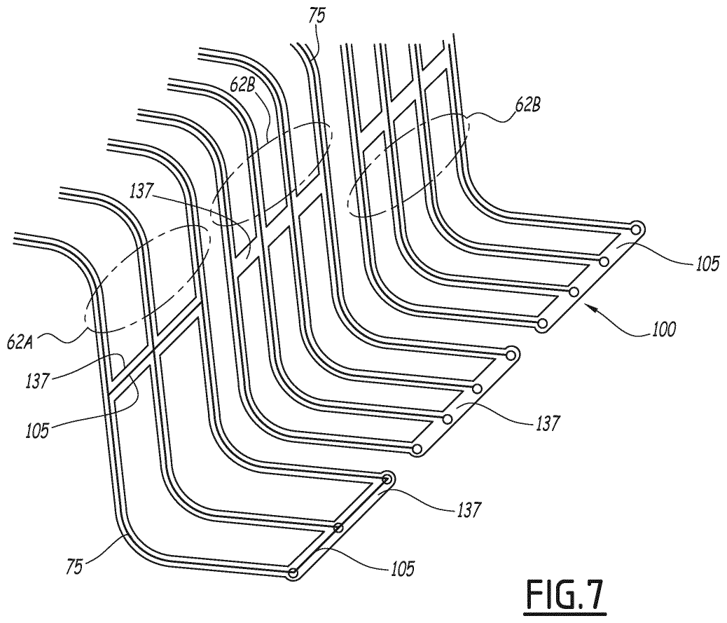

FIG. 7 is a view of a specific mode of implementation of the extremities of the electrodes of FIG. 5,

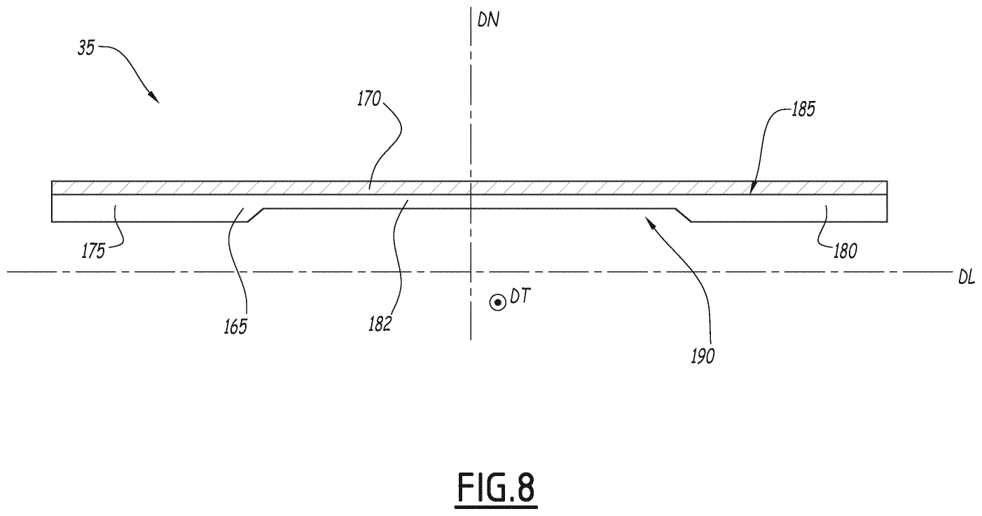

FIG. 8 is a cut-away side view of the electrical connector,

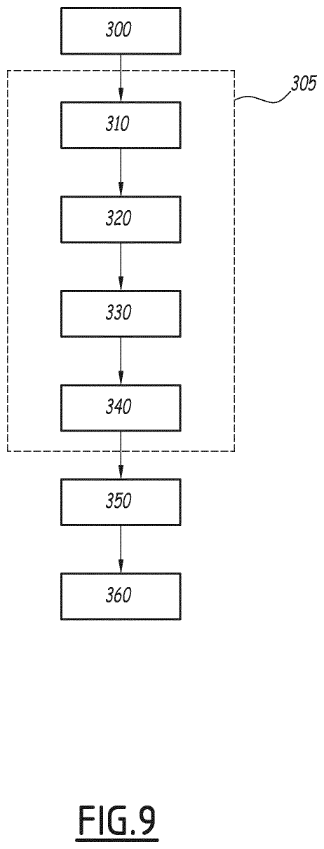

FIG. 9 is an ordinogram of the steps of a method for fabricating the implant of FIG. 1, and

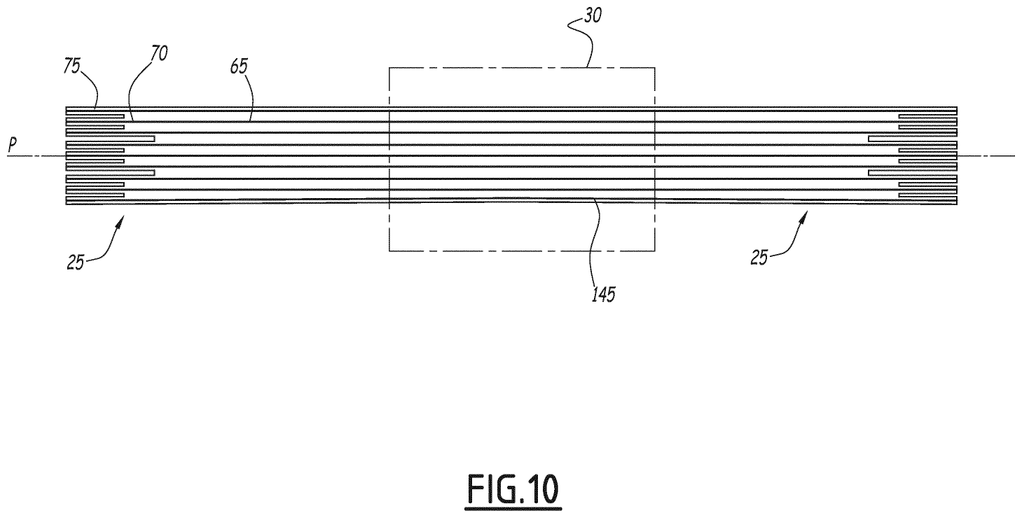

FIG. 10 is a schematics of another example of implant according to the invention.

A first example of an implant 10 and an apparatus 15 are shown on FIG. 1.

The implant 10 is configured to be implanted in a biological tissue of an animal 12.

The biological tissue is, in particular, part of the body of the animal 12.

In a possible variant, the biological tissue of the body is, has been taken from the animal and that is maintained alive artificially.

The animal is, for example, a mammal such as a human being. In a possible variant, the mammal is a rat, a rabbit, a cow, a monkey, a pig, a sheep, a mouse, a dog, or a cat.

In another variant, the animal is an insect, a bird, a reptile, or a fish.

The implant 10 is configured to be at least partially implanted in the body of the animal 12. In particular, at least part of the implant 10 is configured to be placed into an opening of the body, the opening being created by surgery.

The implant 10 is configured to be electrically connected to the biological tissue.

The biological tissue is, for example an organ 20 of the animal's body or part of the organ 20. In particular, the biological tissue is a neural tissue.

The implant 10 is configured to transmit information between the apparatus 15 and the biological tissue. For example, the implant 10 is configured to electrically connect the apparatus 15 to the biological tissue. In this case, the implant 10 is configured to transmit electrical currents from the biological tissue to the apparatus 15, and/or vice-versa.

The organ 20 is, for example, the brain of the animal 12. According to an embodiment, the organ 20 is the brain's cortex. In this case, the implant 10 is a cortical implant.

In a possible variant, the organ is another brain area such as the hippocampus, the thalamus, the hypothalamus, the cerebellum or other brain areas.

In another variant, the organ is a part of the central nervous system such as the retina, the cochlea, the spinal cord, or a part of the peripheral nervous system, or nerves, or the heart, or other organs or biological tissues that may need electrical stimulation or electrically conductive materials to promote its functional activity.

The implant 10 comprises a set of arms 25, an implant body 30, an extension piece 35 and a transfer module 40. As a facultative complement, the implant 10 further comprises at least one reference electrode 45 and/or at least one ground electrode 50.

The set of arms 25 and the implant body 30 are shown in greater details on FIGS. 2 to 5.

In the example shown on FIGS. 2 to 5, the implant 10 comprises six arms 25. However, the number of arms 25 may vary. In particular, the number of arms 25 is comprised between 2 and 16. In a possible variant, the number of arms 25 is strictly greater than 16.

Each arm 25 extends from the implant body along a main direction D. In the example shown on FIG. 2, the main directions D of all arms 25 are coplanar. However, cases where the main directions D of the arms are not coplanar may also be considered.

A lateral direction X is defined for each arm. The lateral direction X is a direction perpendicular to the main direction D and contained in the plane comprising all main directions D.

In the example shown on FIG. 2, the arms extend radially from the body 30. In particular, at least one portion of the body 30 is surrounded in by the arms 25.

For example, an angle α is defined between the main directions of two successive arms 25. Two successive arms 25 are two arms 25 between which no other arm 25 is interposed. The angle α is, for example, identical, within 10 degrees, for each pair of successive arms 25.

In the example shown on FIG. 2, the angle α is, for example, comprised between 0 degrees (°) and 180°. However, it should be noted that the value of the angle α may vary, notably in function of the number of arms 25, or in function of the biological tissue.

The sum of the angles α is, for example, superior or equal to 90 degrees (°), notably superior or equal to 120°. In other words, an angle between the main directions D of the arms 25 which are furthest from each other, is superior or equal to 90 degrees (°), notably superior or equal to 120°.

In a possible embodiment, the sum of the angles α is inferior or equal to 180°.

Each arm 25 has a total length Lt. The total length Lt is measured from the implant body 30 along the main direction D of the arm 25. The total length Lt is, for example, comprised between 5 millimeters (mm) and 50 centimeters (cm), depending on the organ 20 or biological tissue to be implanted. In particular, the total length Lt is identical, for example identical within 10%, for each arm 25.

It should be noted that embodiments wherein the total length Lt varies from one arm 25 to another are also envisioned.

Each arm 25 comprises a bundle 52 of conductive wires 55, also called “main electrodes 55”, and a sheath 60.

Each bundle 52 comprises at least four conductive wires 55. In the example shown on FIGS. 3 and 4, each bundle 52 comprises 10 conductive wires 55. However, the number of conductive wires 55 in each bundle may vary. For example, some bundles 52 may comprise a number of conductive wires 55 different from at the number of conductive wires 55 of at least one other bundle 52. Alternatively, the number of conductive wires 55 in each bundle 52 may be different from 10.

Each bundle 52 of conductive wires 55 comprises at least two subsets 62 of conductive wires 55, for example at least three subsets 62. In the example shown on FIGS. 2 to 4, each bundle 52 comprises three subsets 62 of conductive wires.

Each subset 62 comprises at least two conductive wires 55, for example at least three conductive wires 55.

In the example shown on FIGS. 3 and 4, each bundle 52 comprises two subsets 62 comprising four conductive wires 55 and one subset 62 comprising three conductive wires 55. However, the number of conductive wires 55 in each subset 62 may differ from three or four.

Each conductive wire 55 is configured to electrically connect the biological tissue to the implant body 30. Each conductive wire 55 is electrically conductive.

Each conductive wire 55 is, for example, electrically insulated from all other conductive wires 55. In a possible variant, at least one conductive wire 55 is electrically connected to at least one other conductive wire 55 of the same subset 62. For example, all conductive wires 55 of a same subset 62 are electrically connected to one another.

Each conductive wire 55 extends from the implant body 30 along the main direction D of the corresponding arm 25. It should be noted that variants in which the conductive wires 55 do not extend along the main direction D are also envisioned.

In an embodiment, all conductive wires 55 of a bundle 52 are parallel to each other. For example, a distance Di between successive conductive wires 55 of a same bundle 52 is comprised between 5 micrometers (μm) and 50 μm.

It is to be noted that this distance Di will vary when the implant is placed in a liquid (before its implantation) or in the biological tissue (after its implantation). This distance Di can vary from 0 to several micrometers.

Each conductive wire 55 has a length equal for example to the total length Lt of the arm 25.

Each conductive wire 55 comprises a first portion 65, a second portion 70 and a third portion 75.

Each conductive wire 55 has a lateral dimension Ld, measured for example along the lateral direction X, comprised between 100 nanometers (nm) and 500 μm.

Each conductive wire 55 comprises, for example, a metallic film. The metallic film is configured to transmit an electrical current from the biological tissue to the implant body 30, and vice-versa.

A film is a feature having a thickness, measured along a first direction Z, inferior or equal to one tenth of the dimension of the film along directions perpendicular to the first direction.

Each film comprises, for example, at least one layer of an electrically conductive material such as gold. In the example shown on FIG. 6, each film comprises a stack of layers superimposed along a stacking direction Z. In this case, the first direction Z is the stacking direction Z.

The film has a thickness comprised between 1 nanometer (nm) and 3000 nanometers.

The stacking direction Z is, for example, perpendicular to the plane in which all main directions D are comprised.

The stack comprises, for example, a first layer 80, a second layer 85, a third layer 90 and a fourth layer 95. Each layer 80 to 95 is, for example, perpendicular to the stacking direction Z.

The conductive wire 55 is, for example, delimited along the stacking direction Z by the first layer 80 and the fourth layer 95.

The first layer 80 is made of a first material. The first material is, for example, electrically conductive.

The first material is, for example, a metal such as titanium or another material, for example a semiconductor such as silicon carbide.

The first layer 80 has a thickness, measured along the stacking direction Z, comprised between 1 nm and 3000 nm.

The first layer 80 is, notably, configured to allow for the second layer 85 to be deposited onto the first layer 80.

The second layer 85 is interposed between the first layer 80 and the third layer 90.

The second layer 85 is made of an electrically conductive second material, notably a metal. For example, the second layer 85 is made of gold.

The second layer 85 has a thickness, measured along the stacking direction Z, comprised between 1 and 3000 nm.

Metal layer thicknesses will typically be a compromise between having a low electrical resistance along the whole conductive wire 55, and allowing for acceptable mechanical properties for the implant (notably a high flexibility) and to ensure a continuous electric path along the whole length of the conductive wire 55.

The third layer 90 is interposed between the second layer 85 and the fourth layer 90. The third layer 90 is configured to strengthen the adhesion of the second and fourth layers 85, 90 to the insulating layer 115 compared to the second and fourth layers 85, 90 without any third layer 90.

The third layer 90 is made of a third material. The third material is, for example, electrically conductive.

The third material is, for example, a metal such as titanium or another material, for example a semiconductor such as silicon carbide.

The third layer 90 has a thickness, measured along the stacking direction Z, comprised between 1 nm and 3000 nm.

The fourth layer 95 is configured to protect the first, second and third layers 80, 85, 90 from oxidation inside the animal's body.

The fourth layer 95 is made of a fourth material. The fourth material is, for example, electrically conductive.

The fourth material is, for example, a metal such as platinum.

The fourth layer 95 has a thickness, measured along the stacking direction Z, comprised between 1 nm and 3000 nm.

Optionally, a layer L covers at least partially the fourth layer 95.

The layer L is, for example, made of PEDOT, PEDOT:PSS (also called poly(3,4-ethylenedioxythiophene) polystyrene sulfonate), iridium oxyde, graphene, graphene oxyde, porous graphene, porous graphene oxide, Laminin protein, L1 peptide or RGD peptide, or specific sugars.

Layers L made of PEDOT, PEDOT:PSS and iridium oxyde may be obtained through electrodeposition. Graphene based materials such as graphene sheets or pieces may be part of a suspension that is locally deposited, for example using a pipet, on each conductive wire 55.

The thickness of the layer L is comprised between 1 nm and 50 μm.

It should be noted that examples where the conductive wires 55 are made of a single layer of material are also envisioned. In another possible variant, the conductive wires 55 are cylindrical.

The first portion 65 is electrically connected to the implant body 30.

The first portion 65 of each conductive wire 55 is delimited by the second portion 70 and by the implant body 30.

The first portion 65 is, for example, a parallelepiped. In particular, the first portion 65 is defined by faces that are either perpendicular to the main direction D of the arm 25 containing the conductive wire 55, perpendicular to the stacking direction Z or to the lateral direction X. In particular, a section of the first portion 65 in a plane defined by the directions D and X is a rectangle.

In a variant, the first portion 65 has a wavy shape in a plane defined by the directions D and X of the arm 25 to which the conductive wire 55 belongs. For example, the first portion 65 is defined, along the lateral direction X, by faces that extend, in that plane, along a zigzag or sinusoidal line. For example, the faces include a plurality of facets, successive facets forming between them an angle comprised between 80° and 100°.

The first portion 65 has a first length L1. The first length L1 is, for example, comprised between 10% and 60% of the total length Lt. In particular, the first length L1 is comprised between 30% and 40% of the total length Lt. According to an embodiment, the first length L1 is comprised between 33% and 34% of the total length Lt.

The second portion 70 of each conductive wire 55 is delimited by the first portion 65 and by the third portion 75.

The second portion 70 is, for example, a parallelepiped. In particular, the second portion 70 is defined by faces that are either perpendicular to the main direction D of the arm 25 containing the conductive wire 55, perpendicular to the stacking direction Z or to the lateral direction X. In particular, a section of the second portion 70 in a plane defined by the directions D and X is a rectangle.

In a variant, the second portion 70 has a wavy shape in a plane defined by the directions D and X of the arm 25 to which the conductive wire 55 belongs. For example, the second portion 70 is defined, along the lateral direction X, by faces that extend, in that plane, along a zigzag or sinusoidal line. For example, the faces include a plurality of facets, successive facets forming between them an angle comprised between 80° and 100°.

The second portion 70 has a second length L2. The second length L2 is, for example, comprised between 10% and 60% of the total length Lt. In particular, the second length L2 is comprised between 30% and 40% of the total length Lt. According to an embodiment, the second length L2 is comprised between 33% and 34% of the total length Lt.

The third portion 75 is intended to be electrically connected to the biological tissue.

The third portion 75 of each conductive wire 55 is delimited by the second portion 70.

The third portion 75 has a first extremity connected to the second portion 70 and a second extremity 100 intended to be electrically connected to the biological tissue.

The second extremity 100 is, for example, rounded. In particular, the second extremity is defined along the main direction D by a facet of the third portion, the facet being shaped as a portion of a cylinder.

For example, the facet is a half-cylinder. Smooth shapes such as half-cylinders are less likely to damage cells.

In possible variants, the facet is in the shape of an arrow or of a harpoon. Such shape allow for a better anchoring of the second extremity 100 to the tissue.

As an optional complement, the shape of each facet may be different from the shape of the facet of each other second extremity 100. This will allow for each second extremity 100 to be identified among the other second extremities, even within the tissue, using methods of observation of the local tissue around the second extremity 100 of each third portion 75.

The third portion 75 is, for example, a parallelepiped. In particular, the third portion 75 is defined by faces that are either perpendicular to the main direction D of the arm 25 containing the conductive wire 55, perpendicular to the stacking direction Z or to the lateral direction X. In particular, a section of the third portion 75 in a plane defined by the directions D and X is a rectangle.

According to the embodiment shown on FIG. 5, the third portion 75 has a rounded extremity

In a variant, the third portion 75 has a wavy shape in a plane defined by the directions D and X of the arm 25 to which the conductive wire 55 belongs. For example, the third portion 75 is defined, along the lateral direction X, by faces that extend, in that plane, along a zigzag or sinusoidal line. For example, the faces include a plurality of facets, successive facets forming between them an angle comprised between 80° and 100°.

The third portion 75 has a third length L3. The third length L3 is, for example, comprised between 10% and 60% of the total length Lt. In particular, the third length L3 is comprised between 30% and 40% of the total length Lt. According to an embodiment, the third length L3 is comprised between 33% and 34% of the total length Lt.

According to a possible embodiment, the third portions 75 of at least two conductive wires 55 of at least one subset 62 of a bundle 52 are electrically connected to each other. The third portions 75 are electrically connected by lateral portions 105 of the conductive wire 55, the lateral portions 105 extending for example along the direction X. For example, as shown on FIG. 7, all third portions 75 of at least one subset 62A of conductive wires 55, notably all third portions 75 of each subset 62 of conductive wire 55s of a same bundle 52, are connected to each other by lateral portions 105.

The lateral portions 105 notably help keeping a distance between neighbouring third portions 75 constant event when the implant is inserted in an animal's body or is immersed in a liquid.

For example, each lateral portion 105 links the second extremities 100 of two successive conductive wires 55. In the example shown on FIG. 7, the third portions 75 of successive conductive wire 55 are linked by lateral portions 105 linking the second extremities 100 of the conductive wires 55, and by additional lateral portions 105 interposed between the second extremities 100 and the second portions 70.

However, in any mode of implementation, subsets 62 of conductive wires 55 comprising only conductive wires 55 that are electrically insulated from each other may be present.

Each sheath 60 is independent from the other sheaths 60. In particular, each sheath 60 is fixed to the implant body 30, and is not fixed to any one of the other sheaths 60.

Each sheath 60 is configured to electrically insulate at least part of each conductive wire 55 of the corresponding arm 25 from the outside of the implant 10, notably from the animal's body.

For example, each sheath 60 encases each conductive wire 55 of the arm 25 and defines a single opening 110 for electrically connecting the conductive wire 55 to the biological tissue.

Each sheath 60 is made of an electrically insulating material. This electrically insulating material is, for example, a photosensitive resin such as an epoxy-based resin.

SU-8 is an example of such a photosensitive resin.

SU-8 is composed of Bisphenol A Novolac epoxy that is dissolved in an organic solvent (gamma-butyrolactone GBL or cyclopentanone, depending on the formulation) and up to 10 wt % of mixed Triarylsulfonium/hexafluoroantimonate salt as the photoacid generator).

However, other electrically insulating materials, notably other photosensitive resins may also be envisioned, such as photosensitive polyimide. Other non photosensitive resists as well can be envisioned such as polyimide, parylene, or undoped diamond. Each sheath 60 is, for example, made of superimposed portions of two layers 115, 120 of the electrically insulating material, the conductive wire 55 being interposed between both layers 115, 120. The layers 115, 120 are, notably, superimposed along the Z direction. Each layer 115, 120 has, for example, a thickness, measured along the Z direction, comprised between 50 nm and 50 μm.

The layers 115, 120, are, for example nanostructured, for example by using a plasma etching.

“Nanostructured” refers notably to the presence of nanostructures on the outer faces of the layers 115 and 120.

As an optional complement, the layer L is, also, further, nanostructured.

The nanostructures are, for example, wires or pores. The nanostructures have each, for example, a diameter between 1 nm and 5 μm.

A distance between neighbouring nanostructures is, for example, comprised between 2 nm and 5 μm.

A height of each wire is, for example, comprised between 2 nm and 5 μm.

A depth of each pore is, for example, comprised between, 2 nm and 5 μm.

Nanostructures can vary in size and organization at the surface depending on plasma parameters, such as the time duration of the plasma treatment.

Also, to obtain longer nanostructures, a roughness with a higher aspect ratio, it is possible to deposit a very thin layer of a few nanometers of a metal, such as gold or platinum, before proceeding with the plasma treatment.

This nanostructuration promotes in particular the adhesion of the implant to the biological tissue, improve the biocompatibility, and also limit the corrosion of the implant.

It should be noted that embodiments in which at least one face of the conductive wire 55 and/or the sheath 60 is nanostructured also include embodiments in which the layer 115, the layer 120 and/or the layer L is covered by another layer that is nanostructured.

In an optional variant, each sheath 60 further comprises at least one layer M, for example two layers M.

Each layer M covers at least partially one of the layers 115 and 120. In particular, each layer M covers an outer face of the layer 115, 120. For example, the layers 115 and 120 are interposed between both layers M.

Each layer M is made of a fifth material.

The fifth material has a wettability, with respect to water and to water containing biomolecules, different from the wettability of the electrically insulating material that the layers 115, 120 are made of. They will reduce access of oxidizing species from the biological tissue to the implant surface in order to limit its corrosion. In the meantime, their chemistry is suitable for biological tissue attachment.

The material M is, for example, silicon dioxide.

Silicon dioxide is, for example, deposited using a reaction in suspension where the implant 10 is placed in a solution of TetraEthyl OrthoSilicate.

In a variant, the fifth material is silicon carbide, for example deposited by plasma enhanced chemical vapor deposition.

In a variant, the fifth material is graphene, graphene oxide, porous graphene, or porous graphene oxide. However, other fifth materials, notably carbon-based fifth materials such as carbon nanotubes, may be envisioned.

One way of depositing these graphene-based materials is to have them as sheets or pieces in a suspension in which the layers 115 and 120 are dipped, so that the fifth material covers the insulating layers 115, 120 of the implant.

The addition of this fifth material promotes the adhesion of the implant to the biological tissue, improves the biocompatibility and also limits the corrosion of the implant 10. Carbon based layers further allow the chemical attachment of proteins (such as Laminin) or peptides of adhesion (such as L1 peptide, or RGD peptide), or specific sugars, the attachment being covalent or non-covalent.

The layer M has a thickness comprised between, for example, 2 nm and 5 μm. In another variant, the material M is a Laminin coating, or a L1 peptide coating or

RGD peptide coating to promote the adhesion of the neural tissue.

Each sheath 60 comprises a single proximal portion 125, a set of middle portions 130 and a set of distal portion 135. In particular, each sheath 60 comprises one middle portion 130 for each subset 62 of conductive wire 55 and one distal portion 135 for each conductive wire 55.

As appears in the example shown on FIGS. 2, 3 and 4, the proximal portion 125 divides itself into one middle portion 130 for each subset 62, and the middle portions 130 in turn divide themselves into one distal portion 135 for each conductive wire 55.

As will appear below, in a possible mode of implementation of the invention, the proximal portion 125, the middle portions 130 and the distal portions 135 are integral with each other.

The proximal portion 125 extends from the implant body 30, notably along the main direction D of the arm 25.

In particular, each proximal portion 125 has an extremity connected to the implant body 30 and another extremity connected to the corresponding middle portion 130. The proximal portion 125 extends from the implant body 30 to the corresponding middle portion 130.

According to the example shown on FIG. 3, the proximal portion 125 has a rectangular shape in a plane defined by the directions D and X.

In a possible variant, the proximal portion 125 has a wavy shape in that plane. For example, the proximal portion 125 has sinusoidal side facets defining the proximal portion 125 along the direction X.

The proximal portion 125 encases the first portion 65 of each conductive wire 55 of the bundle 52 of conductive wires 55 corresponding to the arm 25. In particular, the proximal portion solidarizes the first portions 65. In other words, a movement of one of the first portions 65 with respect to the other first portions 65 of the same arm 25 creates, via the proximal portion 125, a force applied to at least one of the other first portions 65.

The proximal portion 125 defines, for example, one duct extending from the implant body 30 to the corresponding middle portion 130 for each conductive wire 55 of the arm 25, the first portion 65 of each conductive wire 55 of the arm 25 being encased in the duct.

The proximal portion 125 has a width, measured along the direction X, comprised between 5 μm and 10 cm.

The proximal portion 125 has a thickness, measured along a direction perpendicular to the main direction D and the direction X, notably the direction Z, comprised between 500 nm and 300 μm.

The proximal portion 125 has a length, measured along the main direction D, equal to the first length L1.

Each middle portion 130 of the sheath 60 of an arm 25 corresponds to one of the subsets 62 of conductive wires 55 of this arm 25. Therefore, the sheath 60 of the arm 25 comprises as many middle portions 130 as subsets 62, one unique middle portion 130 for each subset 62.

Each middle portion 130 is independent from the other middle portions 130. In particular, each middle portion 130 is not fixed to, notably not in contact with, any other middle portion 130. For example, a distance, measured along the direction X, between successive middle portions 130 (i.e middle portions 130 of a same arm 125, between which no other middle portion 130 is interposed), is comprised between 5 μm and 200 μm.

Each middle portion 130 extends from the proximal portion 125, for example along the main direction D of the arm 25. Each middle portion 130 is interposed between the proximal portion 125 and each distal portion 135 of the subset 62 corresponding to the middle portion 125.

In particular, each middle portion 130 has an extremity connected to the corresponding proximal portion 125 and another extremity connected to the corresponding distal portions 135. The middle portion 130 extends between the corresponding proximal and distal portions 125, 135, notably along the main direction D. In the latter case, the middle portion 130 is delimited along the main direction D by the corresponding proximal and distal portions 125, 135.

The middle portion 130 encases the second portion 70 of each conductive wire 55 of the subset 62 of conductive wires 55 corresponding to the middle portion 130. In particular, the middle portion 130 solidarizes the second portions 70 of the subset 62 with one another. In other words, a movement of one of the second portions 70 with respect to the other second portions 70 of the same subset 62 creates, via the middle portion 130, a force applied to at least one of the other second portions 70.

The middle portion 130 defines, for example, one duct extending from the proximal portion 125 to the corresponding distal portion 135 for each conductive wire 55 of the subset 62, the second portion 70 of each conductive wire 55 of the subset 62 being encased in the duct.

The middle portion 130 has a width, measured along the direction X, comprised between 5 μm and 1 cm.

The middle portion 130 has a thickness, measured along a direction perpendicular to the main direction D and the direction X, notably the direction Z, comprised between 500 nm and 300 μm.

According to the example shown on FIG. 3, the middle portion 130 has a rectangular shape in a plane defined by the directions D and X.

In a possible variant, the middle portion 130 has a wavy shape in that plane. For example, the middle portion 130 has sinusoidal side facets defining the middle portion 130 along the direction X.

The middle portion 130 has a length, measured along the main direction D, equal to the second length L2.

Each distal portion 135 is independent from the other distal portion 135. In particular, each distal portion 135 is not fixed to, notably not in contact with, any other distal portion 135. For example, a distance, measured along the direction X, between successive distal portions 135 (i.e distal portions 135 attached to a same middle portion 130, between which no other distal portion 135 is interposed), is comprised between 200 nm and 100 μm.

Each distal portion 135 corresponds to a single conductive wire 55. Each distal portion 135 extends from the middle portion 130 corresponding to the conductive wire 55, for example along the main direction D of the arm 25. However, embodiments wherein at least one distal portion 135 extends along a direction different from the main direction D are also envisioned.

In particular, each distal portion 135 is delimited along the direction along which the distal portion 135 extends, notably along the main direction D, by the corresponding middle portion 130.

The distal portion 135 encases the third portion 75 of the corresponding conductive wire 55.

The distal portion 135 defines, for example, one duct extending from the corresponding distal portion 135, the third portion 75 of the conductive wire 55 being encased in the duct.

The distal portion 135 has a width, measured along the direction X, comprised between 200 nm and 600 μm.

For example, the width of the distal portion 135 is comprised between 200 nm and 30 μm. Such a width allows for a better repopulation and adhesion of cells from the tissue around the implant 10.

In a variant, the width of the distal portion 135 is comprised between 30 μm and 600 μm. Such a width allows for a better mechanical resistance to the handling of the implant during surgery.

The distal portion 135 has a thickness, measured along a direction perpendicular to the main direction D and the direction X, notably the direction Z, comprised between 500 nm and 300 μm.

For example, the thickness of the distal portion 135 is comprised between 200 nm and 30 μm. Such a thickness allows for a better repopulation and adhesion of cells from the biological tissue around the implant 10.

In a variant, the thickness of the distal portion 135 is comprised between 30 μm and 600 μm. Such a thickness allows for a better mechanical resistance to the handling of the implant 10 during surgery.

According to the example shown on FIG. 4, the distal portion 135 is, for example, defined along the direction X by side facets that are perpendicular to this direction X.

In a possible variant, the distal portion 135 has a wavy shape in a plane defined by directions D and X. For example, the distal portion 135 has sinusoidal side facets defining the distal portion 135 along the direction X.

The distal portion 135 has a length, measured along the main direction, strictly superior to the third length L3. For example, the distal portion 135 has a length superior or equal to the sum of the third length L3 and 500 nm. In particular, a difference between the length of the distal portion 135 and the third length L3 is comprised between 50 nm and 50 μm.

According to a possible embodiment, the distal portion 135 of at least two conductive wires 55 of at least one subset 62 of a bundle 52 are linked by a linking portion 137 of the sheath 60.

Each linking portion 137 extends, for example, along the direction X. However, other orientations are possible.

Each linking portion 137 encloses, for example, a corresponding lateral portion 105. However, embodiments wherein at least one linking portion 137 does not enclose any lateral portion 105 are also envisioned, for example if the conductive wires 55 corresponding to the distal portions 135 that are linked by the linking portion 137 are not connected to each other by a lateral portion 105.

On FIG. 7, three subsets of conductive wires 55 are shown. One subset 62A has the third portions 75 of its conductive wires 55 connected to each other by respective lateral portions 105, each lateral portion being encased in a corresponding linking portion 137. Two subsets 62B have their distal portions 135 linked to each other by corresponding linking portions 137, but the corresponding conductive wires 55 are not electrically connected by lateral portions 105.

The distal portion 135 defines the opening 110. In particular, the opening 110 extends along the direction Z from an outer surface of the distal portion 135 towards the third portion 75 of the conductive wire 55.

The opening 110 is, for example, cylindrical. The opening 110 has, in particular, a diameter comprised between 50 nm and 100 μm.

In a variant, the opening 110 extends along the main direction D. For example, the opening 110 extends along the whole distal portion 135, notably having a length equal to the third length L3. In another variant, the opening 110 has a length, measured along the main direction D, superior or equal to the third length L3, notably equal to the sum of lengths L2 and L3, or equal to the sum of lengths L1, L2 and L3.

When the opening 110 extends along the main direction D, a width of the opening 110 is comprised between 50 nm and 100 μm. The opening 110 has a first area value. The first area value is the value of the area of the conductive wire 55 that is left exposed and able to be electrically connected to the biological tissue through the opening 110.

The layer L covers, for example, the portion of the metallic film that is delimited in a plane perpendicular to the Z direction by the opening 110. For example, the layer L is deposited onto the metallic film through the opening 110.

The implant body 30 is configured to transmit an electrical current between each conductive wire 55 and the extension piece 35.

The implant body 30 comprises, for example, an electrical connector 140, a set of electrical conductors 145 and an insulating envelope 150.

In an embodiment, the implant body comprises a main portion 155 and an attachment portion 160.

As will appear below, in possible embodiments, the implant body 30 is integral with the arms 25.

The electrical connector 140 is configured to be electrically connected to the extension piece 35.

The electrical connector 140 is, notably, configured to carry an electrical current between each electrical conductor 145 and the extension piece 35. For example, the electrical connector 140 is formed by a face of the implant body having electrically conductive connection pads to which the extension piece 35 is meant to be electrically connected.

The implant body 30 comprises, for example, one electrical conductor 145 for each conductive wire 55. In a possible variant, the implant body 30 further comprises one electrical conductor 145 for each reference electrode 45 and/or for each ground electrode 50.

The electrical conductor 145 is configured to carry an electrical current between the electrical conductor 140 and the corresponding conductive wire 55, reference electrode 45 or ground electrode 50.

The electrical conductor 145 is, for example, integral with the corresponding conductive wire 55, reference electrode 45 or ground electrode 50. In particular, the electrical conductor 145 is monolithic with the corresponding conductive wire 55, reference electrode 45 or ground electrode 50. Notably, the electrical conductor 145 comprises a stack of layers superimposed along the direction Z.

The insulating envelope 150 is configured to electrically insulate each electrical conductor 145 from the other electrical conductors 145. Additionally, the insulating envelope 150 is configured to electrically insulate each electrical conductor 145 from the outside of the implant body, except from the corresponding conductive wire 55, reference electrode 45 or ground electrode 50 and the electrical connector 140.

The insulating envelope 150 is, for example, integral with, notably monolithic with, the insulating sheath 60 of each arm 25. In particular, the insulating envelope 150 comprises superimposed portions of layers 115, 120 of the electrically insulating material, the electrical conductors 145 being interposed between both layers 115, 120.

When each electrical conductor 145 is integral with the corresponding conductive wire 55 and the insulating envelope 150 is integral with the insulating sheaths, the implant body 30 is integral with the arms 25. However, other modes of implementation wherein the implant body 30 is integral with the arms 25 may also be envisioned.

The main portion 155 is interposed between the attachment portion 160 and the electrical connector 140. The main portion 155 is traversed by each electrical conductor 145.

The attachment portion 160 extends from the main portion 155. The attachment portion 160 is interposed between the main portion 155 and each arm 25. In particular, each arm 25 extends, notably along the arm's main direction D, from the attachment portion 160.

The attachment portion 160 is, for example, shaped as a circular sector. In particular, the attachment portion 160 is defined by an outer face of the attachment portion 160, the outer face being a portion of a cylinder.

The attachment portion 160 has a diameter comprised between 500 μm and 10 cm. In the embodiment shown on FIG. 2, at least one portion of the attachment portion 160 is interposed between two of the arms 25. In particular, the arms 25 extend radially from the attachment portion 160.

The extension piece 35 is shown on FIG. 8.

The extension piece 35 is electrically connected to the electrical connector 140 and to the transfer module 40. The extension piece 35 is configured to carry electrical currents between the electrical connector 140 and the transfer module 40.

The extension piece 35 is, notably, configured to carry an electrical current between each connection pad of the electrical connector 140 and the transfer module 40.

The extension piece 35 comprises a substrate 165 and a set of conductive lines 170. The substrate 165 has, for example, a first extreme portion 175, a second extreme portion 180 and an intermediate portion 182.

The substrate 165 extends between the first extreme portion 175 and the second extreme portion 180. In particular, the substrate 165 is delimited by the first extreme portion 175 and the second extreme portion 180.

According to the embodiment shown on FIG. 8, the substrate 165 extends along a longitudinal direction DL between the first extreme portion 175 and the second extreme portion 180. The substrate 165 is delimited by first extreme portion 175 and the second extreme portion 180 along the longitudinal direction DL.

Additionally, a normal direction DN and a transversal direction DT are defined for the substrate 165. The longitudinal direction DL, the normal direction DN and the transversal direction DT are perpendicular to each other.

The substrate 165 has, for example, a support face 185 perpendicular to the normal direction DN and a back face 190 opposed to the support face 185. The substrate 165 is delimited along the normal direction DN by the support face 185 and by the back face 190.

The substrate 165 is, for example, a board, notably for carrying printed circuits. The substrate 165 is, in another example, a flexible circuit, that was realized through photolithography.

The substrate 165 is made of an electrically insulating material such as polyimide, SU8 resist, parylene or undoped diamond.

The substrate 165 has a total length, measured along the longitudinal direction DL, comprised between 0.5 cm and 40 cm.

The substrate 165 has a width, measured along the transversal direction DT, comprised between 1 mm and 3 cm.

The first extreme portion 175 has a first thickness, measured along the normal direction DN. The first thickness is, for example, comprised between 5 micrometers (μm) and 500 μm, notably comprised between 150 μm and 250 μm, in particular equal to 200 μm, within 10%.

The first extreme portion 175 has a length, measured along the longitudinal direction DL, comprised between 100 μm and 5 mm.

The first extreme portion 175 has a width, measured along the transversal direction DT, equal to the width of the substrate 165.

The second extreme portion 180 has a second thickness, measured along the normal direction DN. The second thickness is, for example, comprised between 5 micrometers (μm) and 500 μm, notably comprised between 150 μm and 250 μm, in particular equal to 200 μm, within 10%.

The second extreme portion 180 has a length, measured along the longitudinal direction DL, comprised between 100 μm and 5 mm.

The second extreme portion 180 has a width, measured along the transversal direction DT, equal to the width of the substrate 165.

The intermediate portion 182 is delimited along the longitudinal direction DL by the first extreme portion 175 and the second extreme portion 180. The intermediate portion 182 is interposed between the first and second extreme portions 175, 180.

The intermediate portion 182 has a width, measured along the transversal direction DT, equal to the width of the substrate 165.

The intermediate portion 182 has a length, measured along the longitudinal direction DL, comprised between 0.5 cm and 40 cm.

The intermediate portion 182 has a third thickness, measured along the normal direction DN, strictly inferior to the first thickness and the second thickness. The third thickness is, for example, comprised between 5 μm and 500 μm, notably equal, within 10%, to 50 μm. In a possible variant, the third portion is equal to at least one, for example both, of the first thickness and second thickness.

The extension piece 35 comprises, for example, one conductive line 170 for each conductive wire 55, and for each reference or ground electrode, 45, 50.

Each conductive line 170 is configured to carry electrical currents between the first extreme portion 175 and the second extreme portion 180.

Each conductive line 170 is configured to be electrically connected to the electrical connector 140 and to the transfer module 40. In particular, each conductive line 170 is configured to carry electrical currents between the electrical connector 140 and the transfer module 40. In that case, the conductive line 170 is, for example, electrically connected to the electrical connector 140 in an area of the first extreme portion 175 and to the transfer module 40 in another area of the second extreme portion 180.

Each conductive line 170 is carried by the support face 185. In particular, each conductive line 170 extends on the support face 185 from the first extreme portion 175 to the second extreme portion 180.

Each conductive line 170 is made of an electrically conductive material, such as copper or gold.

The transfer module 40 is electrically connected to the extension piece 35. In particular, the transfer module 40 is configured to receive electrical currents from each conductive line 170 and/or to inject electrical currents into each conductive line 170. In particular, the transfer module 40 is configured to receive electrical currents from each main, reference or ground electrode 55, 45, 50 via the conductive line 170 connected to this main, reference or ground electrode 55, 45, 50 and/or to inject electrical currents into this main, reference or ground electrode 55, 45, 50 via the conductive line 170 connected to this electrode 55, 45, 50.

In particular, the transfer module 40 is configured to be electrically connected to each conductive line 170, for example via a zero insertion force (Zif) socket of the transfert module 40, the second extreme portion 180 of the extension piece 35 being inserted into the Zif socket so as to electrically connect each conductive line 170 to the transfer module 40.

The transfer module 40 comprises, for example, a communication module 195. The communication module 195 is, for example, able to exchange data with the apparatus 15 through a radiofrequency datalink, such as a Wi-fi, Lo-Ra or Bluetooth link. In this case, the communications module 195 is configured to convert the electrical currents received from the electrodes 45, 50, 55 into data and to transmit the data to the apparatus 15 using the radiofrequency datalink. In a variant, the communications module 195 is configured to receive data from the apparatus 15 using the radiofrequency datalink, to convert the data into electrical currents and to transmit the electrical currents to the electrodes 45, 50, 55 through the extension piece 35 and the implant body 30.

Alternately, the communication module 195 is configured to receive electrical currents from the apparatus 15 through a physical link such as a cable and to transmit the electrical currents to the electrodes 45, 50, 55, and/or to receive the electrical currents from the electrodes 45, 50, 55 and to transmit the currents to the apparatus 15 via the physical link.

In another variant, the communication module 195 is able to exchange data with the apparatus 15 as electrical currents through a physical link and to convert the data into electrical currents transmitted to the electrodes 45, 50, 55, or to receive electrical currents from the electrodes 45, 50, 55, to convert the electrical currents into data and to transmit the data to the apparatus 15.

Each reference electrode 45 extends from the implant body 30, for example from the main potion 155. Each reference electrode 45 extends, for example, along a main direction D of the reference electrode 45.

The main direction D of each reference electrode 45 is, for example, coplanar with the main direction D of each conductive wire 55.

Each reference electrode 45 is made of an electrically conductive material. Each reference electrode 45 is, for example, integral with the implant body 30.

In an embodiment, each reference electrode 45 is made of the same material or materials as each conductive wire 55. For example, each reference electrode 45 comprises the same stack of electrically conductive layer(s) as the conductive wire 55.

Each reference electrode 45 is encased in an electrically insulating cover. The cover is, for example integral with the envelope 150, notably made of the same electrically insulating material.

The cover defines an opening 110 for electrically connecting an exposed conductive zone of the reference electrode 45 to the body of the animal, notably to the biological tissue.

The opening 110 of the reference electrode 45 has a second area value. The second area value is, for example, strictly superior to the first area value.

For example, the opening 110 of the reference electrode 45 is cylindrical, with a diameter strictly superior to the diameter of the opening 110 of each reference electrode 45.

In the embodiment shown on FIG. 2, the second area value of each reference electrode 45 is superior or equal to the first area value of a main electrode 55. For example, the diameter of the opening 110 of each reference electrode 45 is comprised between 50 nm and 1 mm.

Each ground electrode 50 extends from the implant body 30, for example from the main potion 155. Each ground electrode 50 extends, for example, along a main direction D of the ground electrode 50.

The main direction D of each ground electrode 50 is, for example, coplanar with the main direction D of each conductive wire 55.

Each ground electrode 50 is made of an electrically conductive material. Each ground electrode 50 is, for example, integral with the implant body 30.

In an embodiment, each ground electrode 50 is made of the same material or materials as each conductive wire 55. For example, each ground electrode 50 comprises the same stack of electrically conductive layer(s) as the conductive wires 55.

Each ground electrode 50 is encased in an electrically insulating cover. The cover is, for example integral with the envelope 150, notably made of the same electrically insulating material.

The cover defines an opening 110 for electrically connecting an exposed conductive zone of the ground electrode 50 to the body of the animal, notably to a bone of the animal.

The opening 110 of the ground electrode 50 has a third area value. The third area value is, for example, strictly superior to the first area value.

For example, the opening 110 of the ground electrode 50 is cylindrical, with a diameter strictly superior to the diameter of the opening 110 of each conductive wire 55.

In the embodiment shown on FIG. 2, the third area value of each ground electrode 50 is strictly superior to the first area value of a main electrode 55. For example, the diameter of the opening 110 of each ground electrode 50 is comprised between 50 nm and 1 mm.

According to the embodiment shown on FIG. 2, the main directions D of one ground electrode 50 and of one reference electrode 45, together, define an angular sector, the main directions D of each conductive wire 55 being comprised in the angular sector.

The main directions D of the ground and reference electrodes 45, 50 that define the angular sector form, for example, an angle strictly inferior to 180° with each other.

The openings 110 of each conductive wire 55, of each ground electrode 50 and or each reference electrode 45 are, for example, disposed, notably centered each, along a contour C. The contour C is, for example, a portion of a circle. In particular, the contour C is a portion of a circle centered on a point, each main direction D of each main, reference or ground electrode 55, 45, 50 passing through this point.

The diameter of the circle that the contour C forms a portion of is, for example, comprised between 5 mm and 50 cm.

In a possible variant, a distance between the center of each opening 110 and the contour C is strictly inferior to 5% of the diameter of the circle.

However, embodiments wherein the openings 110 are not centered on a contour C that is a portion of a circle may also be envisioned.

The apparatus 15 is configured to allow a user of the apparatus 15 to monitor parameters of the biological tissue, such as electrical parameters of the electrical currents carried from the organ to the transfer module 40, and/or to command electrical currents to be sent to the biological tissue.

For example, the apparatus 15 is configured to receive, from the transfer module 40, values of parameters of the electrical currents carried by the main, reference and/or ground electrodes 55, 45, 50, and to display the received values to the user.

The parameters comprise, for example, electrical intensities of the electrical currents and/or electrical voltages between the main or reference electrodes 45, 55 and one or several ground electrodes 50. As an optional complement, the values include phases and/or frequencies of the intensities and/or voltages.

In a possible variant, the apparatus 15 is configured to receive the electrical currents from the main, reference and/or ground electrodes 55, 45, 50 via the transfer module 40, and to determine the values of the parameters, for example using sensors such as ammeters and/or voltmeters.

In another variant, the apparatus 15 is configured to allow the user to command the emission of electrical currents to the conductive wires 55, for example by the transfer module 40 or by the apparatus 15, and to modify values of the parameters.

The apparatus 15 comprises, notably, a communications module 200, a processor 205, a memory 210, a human-machine interface 215, a generation module 220 and/or an extraction module 225.

The communications module 200 is able to communicate with the communication module 195 of the transfer module 40. For example, the communication module 200 is configured to receive data and/or electrical currents from the communication module 195.

In a possible complement, the communication module 200 is configured to send data and/or electrical currents to the communication module 195.

The human-machine interface 215 is configured to allow the transmission of data between a user and the apparatus 15. The human-machine interface 215 comprises, for example, a display screen and/or a keyboard.

The generation module 220 is configured to command the generation by the communications module 200 of a message and/or of electrical currents, and the sending of this message and/or these electrical currents to the communication module 195.

The generation module 220 is notably configured to command the generation on the basis of data inputted by the user through the human-machine interface 215.

The extraction module 225 is configured to extract data, notably to extract values of the parameters, from a message and/or from electrical currents received by the communication module 200. The extraction module 225 is, further, configured to command the transmission of these data to the user through the human-machine interface 215.

The generation module 220, the communication module 200 and/or the generation module 225 are, for example, constituted at least partially by software instructions stored in the memory 210.

In a possible variant, at least one of generation module 220, the communication module 200 and/or the generation module 225, for example each of generation module 220, the communication module 200 and/or the generation module 225, is formed by dedicated integrated circuits, or by programmable electronic components.

A method for fabricating the implant 10 will now be described.

The method comprises a step 300 for fabricating a layer, a step 305 for depositing at least one layer, a step 350 for depositing a layer and a step 360 for defining at least one opening 110.

During the step 300 for fabricating a layer, a layer of the electrically insulating material is fabricated, for example fabricated onto a substrate such as a silicon wafer or a glass wafer. In particular, during the step 300 for fabricating a layer, the layer 115 of the electrically insulating material is fabricated, in which case the layer to be fabricated is the layer 115.

Notably, the layer 115 is deposited onto the substrate by a photolithography process. In particular, the electrically insulating material is either deposited onto selected areas of the wafer so as to form the layer 115, or deposited onto the wafer and then partially removed so as to leave on the wafer only the portions of the electrically insulating material that define the layer 115.

In one embodiment, the layer of electrically insulating material is fabricated onto a sacrificial layer carried by the substrate. The sacrificial layer is, for example, made of Nickel or Aluminum. The sacrificial layer is, for example, deposited onto the substrate during a step for depositing the sacrificial layer, this step being implemented prior to the step 300.

The step 305 comprises the deposition, onto the layer 115, of at least one layer 80, 85, 90, 95 of an electrically conductive material.

The step 305 comprises, for example, a step 310, a step 320, a step 330 and a step 340. Each of steps 310 to 340 is, for example, performed using a photolithographic process, including notably material deposition or removal, locally or not locally.

During the step 310, the first layer 80 is deposited onto the layer 115.

During the step 320, the second layer 85 is deposited onto the first layer 80.

During the step 330, the third layer 90 is deposited onto the second layer 85.

During the step 340, the fourth layer 95 is deposited onto the third layer 90.

Thus, during steps 310 to 340, each conductive wire 55 is formed. In particular, each conductive wire 55, each reference electrode 45, each ground electrode 50 as well as each electrical conductor 145 are formed by the layers 80, 85, 90 and 95.

During the step 350, a layer of the electrically insulating material is deposited onto the layer 115 and onto at least one electrically conductive layer 80, 85, 90, 95. In particular, a layer of the electrically insulating material is deposited onto the layer 115 and onto each of the electrically conductive layers 80, 85, 90, 95 so as to form the layer 120.

At the end of step 350, the sheath 60 of each arm 25 is formed. In particular, at the end of step 350, each sheath 60, as well as the implant body 30 and the covers of each reference or ground electrode 45, 50 are formed.

During step 360, that can happen before step 350, or after step 350, each opening 110 is defined. For example, each opening 110 is formed by etching away part of one of the layers of electrically insulating material deposited during one of steps 300 and 350, for example by etching away part of layer 120.

The etching is for example performed using an ion beam. Ion beam etching is a method for etching a material using ions accelerated under an electrical voltage to ablate part of a material.

After step 360, the electrical connector 140 is attached to the implant body 30, the extension piece 35 is attached to the electrical connector 140 and then to the transfer module 40 to obtain the complete implant 10. The implant is, for example, released from the wafer by etching of the sacrificial layer.

According to an embodiment, the electrical connector 140 is attached to the extension piece 35 using an anisotropic conductive film and a thermopress. According to another embodiment, the electrical connector 140 is attached to the extension piece 35 by using pick and place equipment. Optionally, an adhesive material is used to attach the electrical connector 140 to the extension piece 35.

A method for implanting the implant 10 will now be described.

The method comprises a step for solidarizing the implant 10 to an instrument, and a step for implanting.

During the step for solidarizing, the implant 10 is solidarized to an instrument. The instrument comprises a set of tools, notably a first tool and a set of second tools.

The first tool is configured to be fixed to the implant body 30. The first tool comprises, for example, at least pincer able to grip part of the implant body 30.

In a possible variant, the first tool is able to grip part of the extension piece 35 so as to prevent a relative movement of the implant body 30 with respect to the first tool.

The first tool comprises, for example, two pincers able each to grip one of the implant body and the extension piece 35. Each pincer is, for example, a block or a sheet of material in which a slit is opened, the surfaces of the block or sheet that define the slit thus acting as jaws between which the implant body 30 and/or extension piece 35 is gripped.

Each pincer is, for example, made of a plastic material such as poly(dimethylsiloxane) (PDMS), also called dimethylpolysiloxane or dimethicone.

Each second tool is configured to be fixed to one arm 25, to a reference electrode 45 or to a ground electrode 50 and to maintain the arm 25, the reference electrode 45 or the ground electrode 50 in a predetermined position with respect to the first tool.

Each second tool comprises, for example, at least one pincer, notably a PDMS pincer, able to grip the arm 25, the reference electrode 45 or the ground electrode 50.

Each second tool is, for example, maintained in position with respect to the first tool by a support structure, notably a metallic support structure. In some embodiment, the support structure is made of an ensemble of wires, for example twisted together.

The support structure comprises, for example, a handle configured to allow a person to handle the instrument.

During the step for implanting, each conductive wire 55 is electrically connected to the biological tissue. For example, for each arm 25, each conductive wire 55 of the arm 25 are fixed to a same insertion device and inserted simultaneously into the biological tissue using the insertion device. In a possible variant, one insertion device is used for each subset 62 of conductive wires 55.

During the step for implanting, each arm 25 is maintained in position by the instrument until the moment when the surgeon detaches the arm 25 from the corresponding second tool in order to connect the conductive wires 55 of the arm 25 to the biological tissue.

The insertion device is, for example, a metallic wire, notably a steel wire, or a semiconductor wire.

The conductive wire, notably the second extremities 100, are for example removably secured to the insertion device. In particular, the second extremities are secured to the insertion device using a soluble material able to dissolve into the biological tissue, such as polylactic coglycolic acid (PLGA) or bio-soluble polyethylene glycol or Poly Capro Lactone, or cellulose, or Chitosan based compounds.

When a soluble material is used, the insertion device is, notably, retracted out of the body during the step for implanting.

The soluble material is, for example, melted and deposited onto the insertion device, and then fixed to one or several corresponding distal portion(s) 135.

Alternately, the soluble material is deposited onto the insertion device by dissolving the soluble material in a solvent and depositing droplets of the solvent on the insertion device and the corresponding distal portion(s) 135, the droplets being then dried to attach the insertion device to each corresponding distal portion(s) 135.

It should be noted that, when the implant 10 is implanted, either only the distal portion 135 (encasing the third portion 75) of each sheath 60, both the distal portion 135 and middle portion 130 (encasing the second portion 70) or all portions 135, 130 and 125 may be inserted inside the animal's body.

In a possible variant, the insertion device is made of a biodegradable material.

The biodegradable material is, for example, polylactic coglycolic acid (PLGA) or bio-soluble polyethylene glycol or Poly Capro Lactone, or cellulose, or silk or Chitosan based compounds.

In this variant, the insertion device is, for example, arrow-shaped. In another embodiment, the insertion device is a wire.

The insertion device is, for example, fabricated by depositing the biodegradable material onto one or several distal portions 135, and later fusing the biodegradable material by heating. The biodegradable material is thus fixed to the distal portion(s) 135 onto which the insertion device is deposited.

In another embodiment, the biodegradable material is dissolved in a solvent and droplets of the solvent are deposited onto the corresponding distal portion(s) 135. The biodegradable material or the solvent droplets is, notably, deposited so as to form the wire- or arrow-shape of the insertion device.

In another embodiment, the biodegradable material can be deposited locally at the distal portion of the implant by using photolithography. For instance, the biodegradable material can be photosensitive such as photosensitive silk material.

When the insertion device is made of a biodegradable material, the insertion device is left inside the biological tissue after implantation and subsequently biodegrades inside this tissue.

The implant 10 allows for a good mechanical resistance of the conductive wires 55 through the division of the arms 25 into a single proximal portion, several subsets of solidarized conductive wire and finally independent conductive wires 55 (corresponding to the distal portions 135 and the third portions 75), while allowing for the conductive wires 55 of different subsets 62 to be connected to different areas of the biological tissue. The implant 10 also allows a high flexibility of the individual distal portions 135 and third portions 75, so as to accommodate relative movements of different parts of the biological tissue.

Furthermore, when the conductive wires 55 of a single arm 25 are implanted in the biological tissue, the density of conductive wires 55 in the tissue is very high. A high density of conductive wires allows a high density of recorded signals or of stimulation sites and therefore a spatio-temporal pattern of recorded spikes containing a higher amount of information, or of highly accurate stimulated neural activity in space and time. This improves the efficiency of implant.

The high density can be achieved by rising the number of electrodes of an implant but also by the surgical insertion of multiple implants in a body.

In known implants, a lot more independent implants must be used to obtain the same high density of electrodes in different areas of the biological tissue. The implant 10 according to the invention thus simplifies the implantation method and improves the reliability of the implantation.

When the main directions D of the arms 25 are coplanar, the implant 10 may easily be fabricated using photolithographic processes. This is especially true when the implant body 30 is integral with the arms 25.

The radial extension of the arms 25 from the implant body 30, notably around the attachment portion 160, is particularly adapted to implant the conductive wires 55 in different areas of a single cerebral hemisphere. When the angles between successive arms 25 are equal to one another, the implant 10 allows for a good repartition of the conductive wires 55 on the whole hemisphere.

Using proximal portions 125, distal portions 135 and middle portions 130 having similar length, notably comprised between 30% and 40% of the total length Lt of the arm 25 allows for a good compromise between a good mechanical resistance and a good ability of the different subsets 62 to be implanted in different areas of the biological tissue.

Using lateral portions 105 allows the implant to record the activity of a single cell, or to stimulate a single cell, with a plurality of conductive wires 55. This allows for a more precise measure of the cell's signal, notably by enabling to discriminate the signal from potential artifacts by comparing the signals measured by the different conductive wires 55 connected to this cell.

Linking portions 137 allow for an increased mechanical resistance of a subset 62, while allowing a high level of flexibility of the individual third portions 75 and distal portions 135.

Conductive wires 55 comprising a metallic film with a thickness between 1 nm and 3000 nm allows for the implant 10 to be flexible enough and to reduce the risk of mechanical breakage.

Using an extension piece 35 having extremities thicker than the intermediate portion allows for ensuring a good fixation of the intermediate piece 35 to the implant body 30 while allowing for a good flexibility, and thus reducing the risk of breakage of the implant body 30 and/or the arms 25.

The presence of the reference electrodes 45 and ground electrodes 50 in the implant 10, notably integral with the implant body 30, makes the implantation method easier and suppresses the need to add these electrodes 45, 50 separately.

The instrument used for the step for solidarizing the implant 10 allows the arms 25 to be efficiently managed during the implantation, thus reducing the risk of breaking of one arm 25.

Using a method for fabricating the implant 10 comprising the steps 300, 310 and 350 allows for the use of well-controlled processes such as photolithographic processes, thus ensuring good reliability of the fabrication.

When the openings 100 are defined by ion-beam etching, the area of the main, reference or ground electrode 55, 45, 50 that is left exposed by the opening 110 has a surface roughness, resulting of the ion-beam etching, that allows for an improved electrical connection of the biological tissue to the electrode 45, 50, 55.

It should be noted that, although the portions 65, 70, 75 of the conductive wires 55 and 125, 130, 135 of the sheaths 60 are described above as extending along a single main direction D of the corresponding arm 25, embodiments wherein at least two of these portions 65, 70, 75, 125, 130, 135 form a non-zero angle with each other, or event are curved, are also envisioned as part of the invention.

A second example of implant (10) will now be described. Elements identical to the first example are not described again. Only the differences are highlighted.

At least one conductive wire 55 of one arm 25 is electrically connected through the implant body 30 to a conductive wire 55 of another arm 25. In particular, these conductive wires 55 are both electrically connected to a same electrical conductor 145 of the implant body 30.

For example, the implant comprises at least one pair of arms 25, each conductive wire 55 of each arm 25 of each pair being electrically connected to a respective conductive wire 55 of the other arm 25 of the pair. However, the number of arms 25 may vary.

As shown on FIG. 10, the implant body 30 is, for example, interposed between both arms 25 of each pair of arms 25.

For example, each arm 25 extends along a main direction D, the main directions of the arms 25 being parallel to each other. Each arm 25 of each pair of arms 25 extend along a main direction D common to both arms 25 of the pair.

In the second example, each electrical conductor 145 of the implant body 30 extends, for example, along the main direction D corresponding to the conductive wires 55 to which the electrical conductor 145 is electrically connected.

In the second example, the implant 10 is, for example, devoid of extension piece 35 and of transfer module 40. For example, each third portion 75 of each arm 25 is electrically connected to one area of the biological tissue while each third portion 75 of the other arm 25 of the same pair of arms 25 is electrically connected to another portion of the biological tissue.

The second example of implant 10 may be used to connect electrically different areas of a biological tissue or of different biological tissues, for example to restore electrical contact between two areas of biological tissue that have been separated by a cut caused by an accident. The advantages of the second example are identical to the advantages of the first example.

1. An implant, adapted to be implanted at least partially in a biological tissue of an animal, comprising an implant body and a set of electrically conductive wires, each conductive wire comprising a first portion electrically connected to the implant body, a second portion and a third portion configured to form an electrical connection to the biological tissue, the second portion being interposed between the first portion and the third portion,
wherein the implant comprises a set of arms wherein each arm comprises an electrically insulating sheath and a bundle of said electrically conductive wires, each bundle comprising at least two subsets of electrically conductive wires, each subset comprising at least two electrically conductive wires, each sheath having a single proximal portion, a set of middle portions and a set of distal portions, each middle portion corresponding to a subset of electrically conductive wires, each distal portion corresponding to a single conductive wire,
the single proximal portion of a each sheath extending from the implant body and encasing the first portion of each conductive wire of the bundle, each middle portion of the sheath extending from the proximal portion and encasing the second portion of each conductive wire of the corresponding subset, and each distal portion of the sheath extending from a middle portion and encasing the third portion of a single conductive wire of the bundle.

2. The implant according to claim 1, wherein each arm extends from the implant body along a main direction, the main directions of the arms being coplanar.

3. The implant according to claim 2, wherein each arm extends radially from the implant body, an angle between the main directions of a pair of successive arms being notably equal for each pair of successive arms.

4. The implant according to claim 1, wherein the implant body comprises a main portion and an attachment portion, the attachment portion extending from the main portion, each arm extending from the attachment portion, the attachment portion being shaped as a circular sector.

5. The implant according to claim 1, wherein each arm comprises at least three subsets of conductive wires, each subset comprising at least three conductive wires.

6. The implant according to claim 1, wherein a length of the proximal portion, a length of the middle portion and a length of the distal portion of each sheath are each comprised between 30 percent and 40 percent of a total length of the arm.

7. The implant according to claim 1, wherein:
each conductive wire of at least one subset is electrically connected to at least one other conductive wires of the subset, notably by at least one electrical conductor linking the third portions of the conductive wires; and/or
the distal portion of at least one sheath is linked to a distal portion of at least one other sheath of the same subset by a linking portion of the sheath, the linking portion being fixed to both distal portions linked by the linking portion; and/or
a face of at least one conductive wire or of at least one sheath is nanostructured.

8. The implant according to claim 1, wherein each conductive wire comprises a metallic film having a thickness of from 1 nanometer to 3000 nanometers.

9. The implant according to claim 1, comprising an electrical connector configured to be electrically connected to a device distinct from the implant body, the implant body comprising electrical conductors configured to connect each conductive wire to the electrical connector.

10. The implant according to claim 9, further comprising an extension piece comprising a substrate and conductive lines supported by the substrate the substrate comprising a first extreme portion, a second extreme portion and an intermediate portion interposed between the first extreme portion and the second extreme portion the extension piece being connected to the electrical connector at the first extreme portion, each conductive line being configured to carry an electrical current between the first extreme portion and the second extreme portion, a thickness of the intermediate portion being inferior or equal to the thickness of the first extreme portion and inferior or equal to the thickness of the second extreme portion.

11. The implant according to claim 1, wherein the implant body is integral with the arms.

12. The implant according to claim 1, wherein at least one conductive wire of one arm is electrically connected, through the implant body to a conductive wire of another arm.

13. An ensemble comprising an implant according to claim 1 and an instrument comprising a set of tools, the set of tool comprising a first tool fixed to the implant body and, for each arm, a second tool configured to maintain the arm in a predefined position relative to the implant body.

14. A method for fabricating an implant according to claim 1, wherein each sheath is made of an electrically insulating first material, the method comprising:
a step for fabricating a first layer made of the electrically insulating first material,
a step for depositing, onto the first layer, at least one second layer of an electrically conductive second material to form the electrically conductive wires, and,
a step for depositing, onto the first and second layers a third layer of the electrically conductive first material so as to form each sheath.

15. The method according to claim 14, further comprising a step for etching away part of the third layer so as to define, for each conductive wire, an opening for electrically connecting the conductive wire to the cortex, the part of the third layer being etched away using notably an ion beam.

16. A method for implanting an implant, the method comprising a step of implanting, in a biological tissue of an animal, at least one implant according to claim 1.
